(19) United States
(12) Patent Application Publication
(10) Pub. No.: US 2024/0315858 A1
(43) Pub. Date: Sep. 26, 2024

(54) DILATOR AND DUAL WIRE LUMEN SYSTEMS

(71) Applicant: Merit Medical Systems, Inc., South Jordan, UT (US)

(72) Inventors: Michael Adams, Bluffdale, UT (US); Joanne Hislop, American Fork, UT (US)

(21) Appl. No.: 18/612,628

(22) Filed: Mar. 21, 2024

Related U.S. Application Data
(60) Provisional application No. 63/629,947, filed on Mar. 24, 2023.

(51) Int. Cl. A61F 2/954; A61F 2/958; A61M 25/09

(52) U.S. Cl. CPC A61F 2/954; A61F 2/958; A61M 25/09

(57) ABSTRACT

Dual-wire lumen systems and methods of deployment for the same are discussed herein. Methods for deployment of dual-wire lumen systems include inserting a dilator system through a first branching portion of a branching stent prosthesis which includes the first and a second branching portions and a trunk portion. The dilator system includes a dilator and a first guidewire. The method further includes directing the dilator system towards an intersection between the first branching portion, the second branching portion, and the trunk portion of the branching stent prosthesis and advancing a distal end of the dilator toward the second branching portion. The method further includes advancing, at the intersection, a distal end of the first guidewire along the trunk portion through a port located on a sidewall of the dilator. The method further includes retracting the dilator through the first branching portion.

### RELATED APPLICATIONS

This application claims priority to U.S. Provisional Application No. 63/629,947, filed on Mar. 24, 2023 and titled “Dilator and Dual Wire Lumen Systems,” which is hereby incorporated by reference in its entirety.

### TECHNICAL FIELD

The present application relates to stent prostheses, guidewires, catheters and methods of using the same.

### BRIEF DESCRIPTION OF THE SEVERAL VIEWS OF THE DRAWINGS

The embodiments disclosed herein will become more fully apparent from the following description and appended claims, taken in conjunction with the accompanying drawings. The drawings depict only typical embodiments, which embodiments will be described with additional specificity and detail in connection with the drawings in which:

FIG. 1 illustrates a branching stent prosthesis with a guidewire according to embodiments herein.

FIG. 2A illustrates a dual-wire lumen system 200 with a first configuration 201a within a branching stent prosthesis 100, according to embodiments herein FIG. 2B illustrates the dual-wire lumen system 200 with a second configuration 201b within the branching stent prosthesis 100, according to embodiments herein.

FIG. 2C illustrates the dual-wire lumen system 200 with a third configuration 201c within the branching stent prosthesis 100, according to embodiments herein.

FIG. 3 illustrates a branching stent prosthesis deployed within a branching implant site, according to embodiments herein.

FIG. 4 illustrates a branching stent prosthesis and a dual-wire lumen system that includes a guide sheath, according to embodiments herein.

FIG. 5 illustrates a method, according to embodiments disclosed herein.

### DETAILED DESCRIPTION

Dilator systems and/or dual-wire lumen systems may be configured to deliver a guidewire from one branch of a branching stent prosthesis that is deployed within a patient's body to another branch. Such systems may be configured to be advanced from an insertion site external to an anatomical system to stent prosthesis internal to the anatomical system. A dual-wire lumen system may be deployed from an insertion site into a branching stent prosthesis. Such systems may be configured to deliver one guidewire with the assistance of another guidewire.

Stent prostheses may be deployed in various body lumens for a variety of purposes. Stent prostheses may be deployed, for example, in the arterial system for a variety of therapeutic purposes including the treatment of occlusions within the lumens of that anatomical system. The current disclosure may be applicable to stent prostheses designed for the central venous system, peripheral vascular system, abdominal aortic aneurism system, bronchial system, esophageal system, biliary system, or any other system of the human body. Further, the present disclosure may equally be applicable to other prosthesis such as grafts.

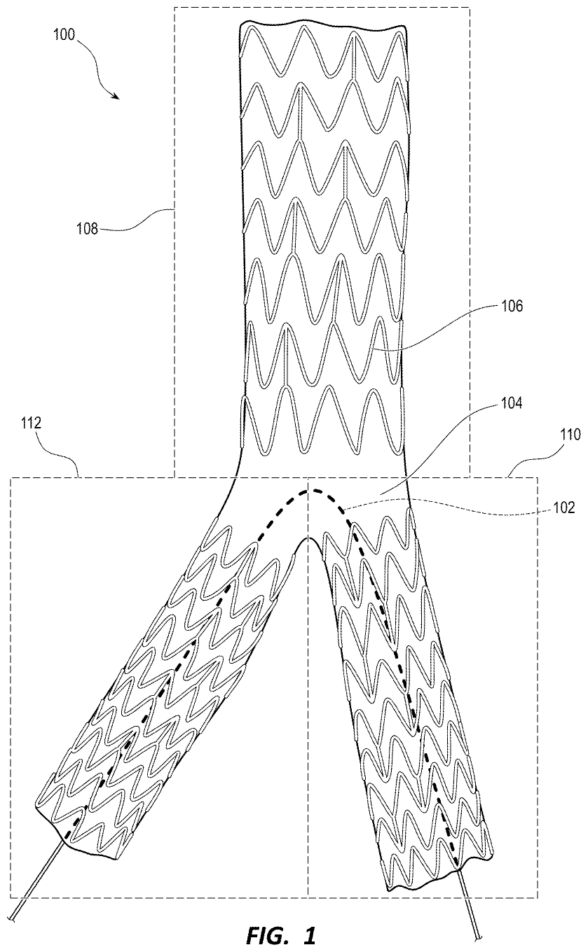

FIG. 1 illustrates a branching stent prosthesis 100 with a guidewire 102 according to embodiments herein. In the illustrated embodiment, the branching stent prosthesis 100 is partially composed of a wire or laser cut stent 106. In some embodiments, the wire or laser cut stent 106 is configured to be expandable to a deployed position as illustrated from an unexpanded/un-deployed configuration (e.g., via the inflation of one or more balloons from within the branching stent prosthesis 100) after the branching stent prosthesis 100 is delivered to a desired location. The wire or laser cut stent 106 may be formed of any suitable material, such as nickel-titanium alloy, stainless steel, cobalt-chromium, platinum, polymers, etc. The wire or laser cut stent 106 may have a zig-zag pattern, a wave pattern, or any other suitable pattern. The wire or laser cut stent 106 may be pre-formed or formed corresponding to a tubular body 104. The material, pattern, and wire diameter of a wire or laser cut stent 106 that is a wire stent, or the wall thickness and strut width of a wire or laser cut stent 106 that is a laser cut stent, may be configured to provide a chronic radial outwardly directed force and a resistance to a radial inwardly directed force. For a non-self-expanding design the deployed wire or laser cut stent 106 may be configured to provide a radial stiffness and radial strength to resist a local or radial inwardly directed force.

The branching stent prosthesis further includes the tubular body 104. The tubular body 104 may be formed of a variety of materials and/or layers of materials, including biocompatible materials that are resistant to passage of fluid through a wall of the tubular body 104. For example, the tubular body 104 may be formed of polyethylene terephthalate, polyurethane, silicone rubber, nylon, fluoropolymer, polyester, etc. A thickness of the wall may range from about 0.025 mm to about 0.5 mm.

In certain embodiments, the wall of the tubular body 104 may be impermeable to tissue cell ingrowth into and/or tissue cell migration through the wall, for example, to prevent or discourage stenosis of the tubular body 104. Additionally or alternatively, in some embodiments, the wall of the tubular body 104 can be impermeable to fluid such that fluid is prevented from leaking from the inside of the branching stent prosthesis 100 to the exterior of the branching stent prosthesis 100 and into surrounding tissue. In some embodiments an interior surface of the wall may include serially deposited fibers of polytetrafluoroethylene (PTFE) to resist fibrin deposition and platelet adhesion on the surfaces.

Note that in embodiments herein, stent prostheses are illustrated as having tubular bodies (such as the tubular body 104 of FIG. 1). However, it will be understood that expandable branching stent prostheses using a wire scaffold, framework, or stent without a tubular body fall within the scope of the disclosure.

As illustrated herein, the branching stent prosthesis 100 may be for deployment at a branching implant site within a body having a trunk, a first branch, and a second branch. Accordingly, the branching stent prosthesis 100 includes a trunk portion 108, a first branching portion 110, and a second branching portion 112. The trunk portion 108, the first branching portion 110, and the second branching portion 112 are joined (e.g., meet) at an intersection. For deployment of the branching stent prosthesis 100 at the branching implant site, the trunk portion 108 of the branching stent prosthesis 100 is used in/at the trunk of the branching implant site, the first branching portion 110 is deployed in/at the first branch of the branching implant site, and the second branching portion 112 is deployed in/at the second branch of the branching implant site.

The guidewire 102 extends between the first branching portion 110 and the second branching portion 112. In other words, the guidewire 102 passes through the second branching portion 110 and out of the second branching portion 112, and may be configured to extend past out of the first branching portion 110 and the second branching portion 112. The guidewire 102 may be positioned within the branching stent prosthesis 100 prior to or during deployment of the branching stent prosthesis 100 to assist in deployment of the branching stent prosthesis 100 to the branching implant site. As such, the guidewire 102 may extend back through a branch of the branching implant site and run to/through an insertion site though which a practitioner has delivered the branching stent prosthesis into the body (e.g., using one or more delivery systems, such as a delivery catheter system in some embodiments). In some embodiments, the guidewire may be termed a floss wire as it runs from one branch to the other and may extend from the branching stent prosthesis 100 to access sites corresponding to each side of the vasculature. Guidewire 102 may also be termed a branch guidewire due to its location.

The guidewire 102 may be configured to exit the opposite branch of the branching implant site such that a length of the guidewire 102 extends to/through an incision, such that the practitioner can have access to the guidewire 102 from two separate and distinct locations of the body (subsequently providing access to the second branching portion 112 and the first branching portion 110 of the branching stent prosthesis 100). In some embodiments, this allows the practitioner to deploy one or more systems along either end of the guidewire 102 into the branching stent prosthesis 100.

The branching stent prosthesis 100 may be deployed within a branching implant site within the body (e.g., within a vascular system of the body). For example, the vascular system may be an aortic/iliac bifurcation. In some embodiments, the branching stent prosthesis 100 may be deployed within any branching implant site that includes a first branching portion, a second branching portion, and a trunk portion.

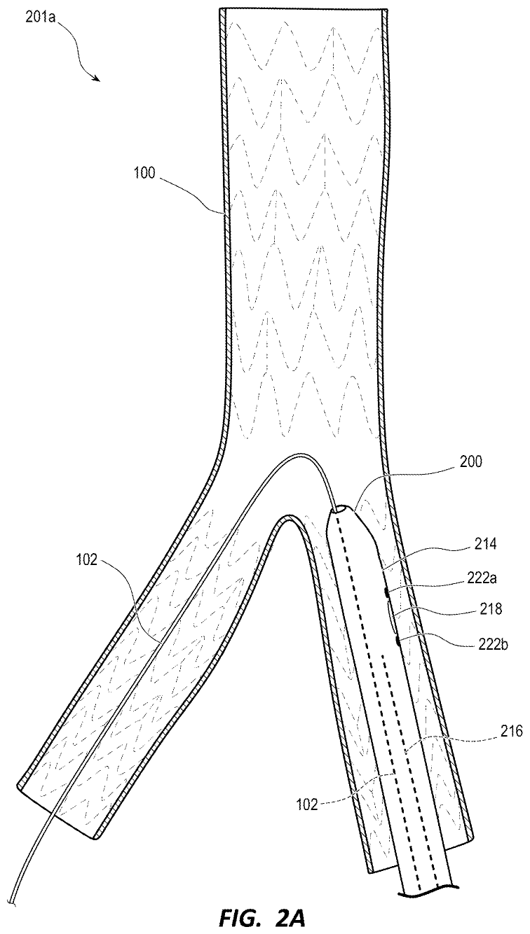

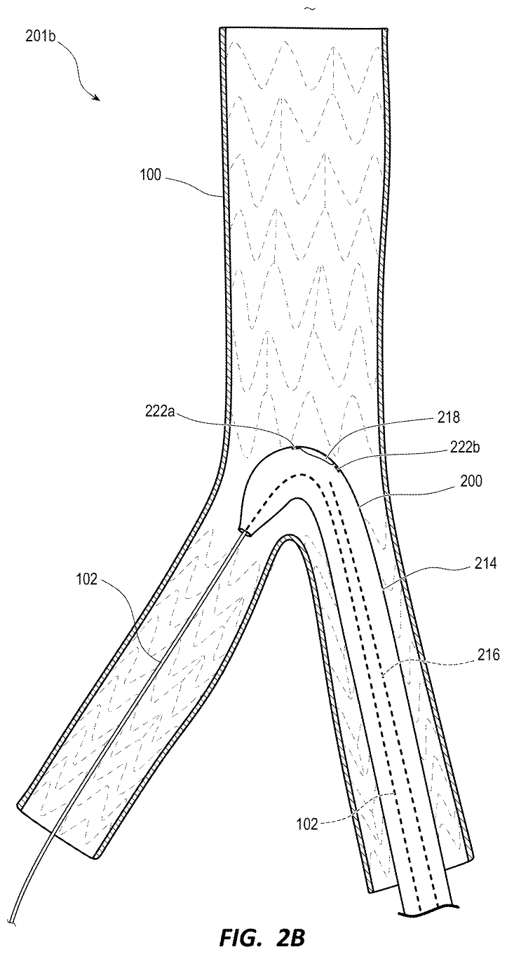

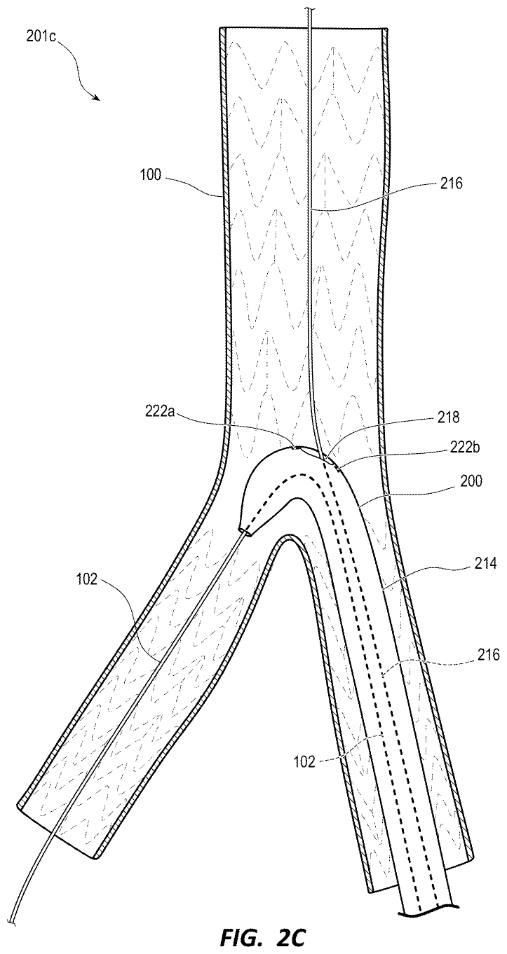

FIG. 2A illustrates a dual-wire lumen system 200 with a first configuration 201a within a branching stent prosthesis 100, according to embodiments herein. The dual-wire lumen system 200 includes a dilator 214 and a guidewire 216. In some embodiments, the dual-wire lumen system further includes the guidewire 102. The guidewire 216 is located within the dilator 214 and runs parallel to the dilator 214 within the first branching portion 110 of the branching stent prosthesis 100. The dilator 214 includes a port 218 and a distal end of the dilator 214 has an opening 220 through which the dilator 214 can be advanced and/or retracted along the guidewire 102.

The port 218 is an opening (e.g., a hole, cutout, slit, incision, or the like) within a sidewall of the dilator 214 that is sufficiently large to allow the guidewire 216 to pass through it. The port 218 may be located adjacent the distal end of the dilator 214 proximal of the distal tip of the dilator 214. In particular, the port 218 may be located closer to the distal end of the dilator 214 than to a proximal end of the dilator 214. As further detailed below, the port 218 may be proximally offset from the distal tip of the dilator 214 such that the port 218 is aligned with a trunk portion of a stent graft when the distal tip of the dilator 214 is partially disposed in a second leg of a stent graft and the proximal portion of the dilator 214 disposed in a first leg of a stent graft.

In some embodiments, one or more edges of the port may be indicated by various means, such as with radiopaque (or radiodense) markers (e.g., a substance which is opaque to x-rays or similar electromagnetic radiation), such as radiopaque markers 222a and 222b as indicated in FIG. 2. The radiopaque markers may be indicated as spots, rings, bands, etc. about the port 218. Radiopaque markers allow the position of the port 218 to be tracked during a procedure. In some embodiments, the dilator 214 may include a radiopacifier to enhance visualization and tracking of the dilator 214 and/or the port 218 during deployment. In some embodiments, the distal end of the dilator 214 may include a radiopaque marker, rather than the port 218, and a distance between the distal end and the port 218 is known in order to allow for tracking of the location of the port 218. In some other embodiments, there may be radiopaque markers on the distal end of the dilator 214, around the port 218, and/or other locations along the dilator 214.

As depicted in FIGS. 2A-2C, the dual-wire lumen system 200 may be configured to be advanced through bends and/or turns within the branching stent prosthesis 100. The dual-wire lumen system 200 may be inserted through the first branching portion 110 of the branching stent prosthesis 100. Within the first branching portion 110, the guidewire 102 and the guidewire 216 are located within the dilator 214 and a distal end of the guidewire may be positioned at a similar distance from the distal end of the dilator 214 as the port 218. In the depicted configuration 201a (e.g., prior to the dilator 214 being advanced through bends and/or turns within the branching stent prosthesis 100), the guidewire 216 is fully enclosed within the dilator 214. The distal end of the dilator 214 and the distal end of the guidewire 216 are advanced along the guidewire 102 which is stationary. The dilator 214 and the guidewire 216 are directed along the guidewire 102 towards the intersection between the trunk portion 208, the first branching portion 110, and the second branching portion of the branching stent prosthesis 100.

FIG. 2B illustrates the dual-wire lumen system 200 with a second configuration 201b within the branching stent prosthesis 100, according to embodiments herein. At or near the intersection, the distal end of the dilator 214 is advanced (e.g., curved as a result of being directed by the guidewire 102) towards the second branching portion 112 while a distal end of the guidewire 216 is advanced (e.g., directed) through the port 218 and along the trunk portion 108, such that the dilator 214 and the guidewire 216 no longer run parallel. In other words, the dilator 214 and the guidewire 216 split into the second branching portion 112 and the trunk portion 108, respectively. The guidewire 216 may be advanced into the trunk portion 108 by means of a practitioner manipulating a proximal end of the guidewire 216. The advancing of the guidewire 216 may occur when the port 218 becomes directed such that it opens towards the trunk portion 108. The guidewire 216 may be advanced when it is determined that the port 218 is directed within the trunk portion 108, for example by means of the radiopaque markers 222a and 222b. It is worth noting that within the first branching portion 110, the proximal portions of the dilator 214 and the guidewire 216 are still parallel, and the corresponding portion of the guidewire 216 is still located within the dilator 214.

FIG. 2C illustrates the dual-wire lumen system 200 with a third configuration 201c within the branching stent prosthesis 100, according to embodiments herein. Once the dilator 214 and the guidewire 216 are separated at the intersection, the dilator 214 may be held stationary, while the guidewire 216 may continue to be advanced through the port 218 and up the trunk portion 108 of the branching stent prosthesis 100, such that a practitioner may be able to access the distal end of the guidewire 216 via the trunk portion 108 (e.g., via an incision, opening, or the like in the body which provides access to the portion 108 via a trunk of the branching implant site. The guidewire 216 may continue to be advanced up and along the trunk portion 108 until the distal end reaches a desired location.

Once the guidewire 216 is positioned at the desired location, the dilator 214 may be retracted along the guidewire 102 back through the first branching portion 110 of the branching stent prosthesis 100. The dilator 214 is retracted in such a manner that there is little to no translational dependency between the dilator 214 and the guidewire 216, such that the guidewire 216 remains in place extending between the first branching portion 110 and the trunk portion 208, while the dilator 214 is entirely extracted. In some embodiments, this may be achieved by constructing the dilator 214 from a sufficiently pliable or flexible material, in comparison to constructing the guidewire 216 from a more rigid material. In other embodiments, this may be achieved by constructing the dilator 214 and the guidewire 216 from the same material, but such that they have different durometers. The dilator 214 and/or the guidewire 216 can be constructed from a material, such as polypropylene, polyethylene, nylon, Pebax, or the like. The dilator 214 and the guidewire 216 can be constructed to have an appropriate flexibility or stiffness (e.g., durometer) in regards to the particular material (e.g., the pliability of the structure can be varied even without changing the material).

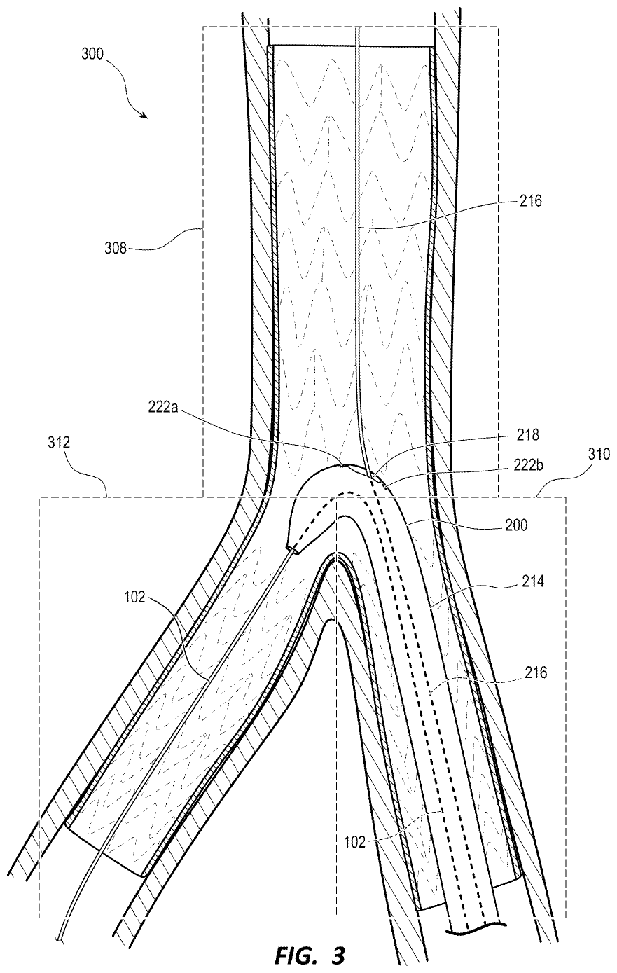

FIG. 3 illustrates a branching stent prosthesis 100 deployed within a branching implant site 300, according to embodiments herein. When the branching stent prosthesis 100 is deployed within the branching implant site 300, it can provide stenting at/through the branching implant site within the anatomical system in which it is deployed.

The branching implant site 300 may be a portion of an anatomical system that includes a trunk 308, a first branch 310, and a second branch 312 that are in mutual fluid communication. The branching stent prosthesis 100 is deployed such that the trunk portion 108 is located within the trunk 308, the first branching portion 110 is located within the first branch 310, and the second branching portion 112 is located within the second branch 312 of the branching stent prosthesis 100 and the branching implant site 300, respectively. In some embodiments, the branching implant site 300 is the aorto-illiac bifurcation in the arterial system.

In some embodiments, the branching stent prosthesis 100, once deployed, provides an appropriate channel for desired liquid flow through the branching implant site 300. It may be that the region around the branching implant site 300 is diseased, misshapen, and/or damaged, and that the deployment of the expandable branching stent prosthesis 100 in the illustrated manner can correct and/or ameliorate attendant issues.

As illustrated, the branching implant site 300 of FIG. 3 is used within an anatomical system that is a cardiovascular system. However, it should be noted that while examples herein describe (and figures herein illustrate) the deployment of branching stent prostheses and dual-wire lumen systems within cardiovascular systems, the cardiovascular anatomical context is given by way of example and not by way of limitation. It will be understood that deployment systems analogous to those described in relation to disclosure herein may be used to deploy dual-wire lumen systems within appropriate branching stent prostheses with/at branching implant sites of other anatomical systems, and that corresponding methods for using such deployment systems to deploy corresponding dual-wire lumen systems that are analogous to those methods described herein could be used in those other anatomical contexts

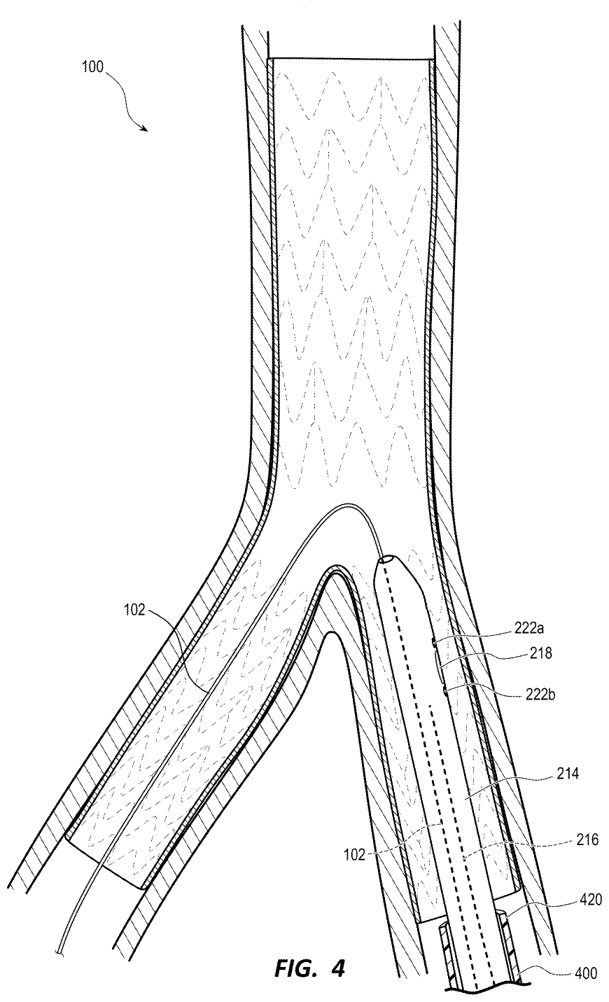

FIG. 4 illustrates a branching stent prosthesis 100 and a dual-wire lumen system 400 that includes a guide sheath 420, according to embodiments herein. The dual-wire lumen system 400 may be similar to the dual-wire lumen system 200, as noted by similar reference numbers, except that in addition to the dilator 214 and the guidewire 216, the dual-wire lumen system 400 further includes a guide sheath 420.

In some embodiments, the guide sheath 420 may be implemented to surround or house the dilator 214, the guidewire 216, and a portion of the guidewire 102 until the distal end of the dilator 214 reaches the interior of the first branching portion 110 of the branching stent prosthesis 100. The guide sheath 420 may be configured to transport the dilator 214 and the guidewire 216 from an insertion site connecting an exterior of a body to a branch (e.g., the branch 310 as illustrated in FIG. 3) and to a corresponding branching portion (e.g., the first branching portion 110 of the branching stent prosthesis 100) along the guidewire 102. The guide sheath 420 may be configured to prevent snagging of the dual-wire lumen system 400 during placement. Once the dilator 214 has been positioned at the desired location (e.g., depth within the first branching portion 110 of the branching stent prosthesis 100), the guide sheath 420 may be retracted back along the dual-wire lumen system 400 and the guidewire 102 and out of the first branching portion 110.

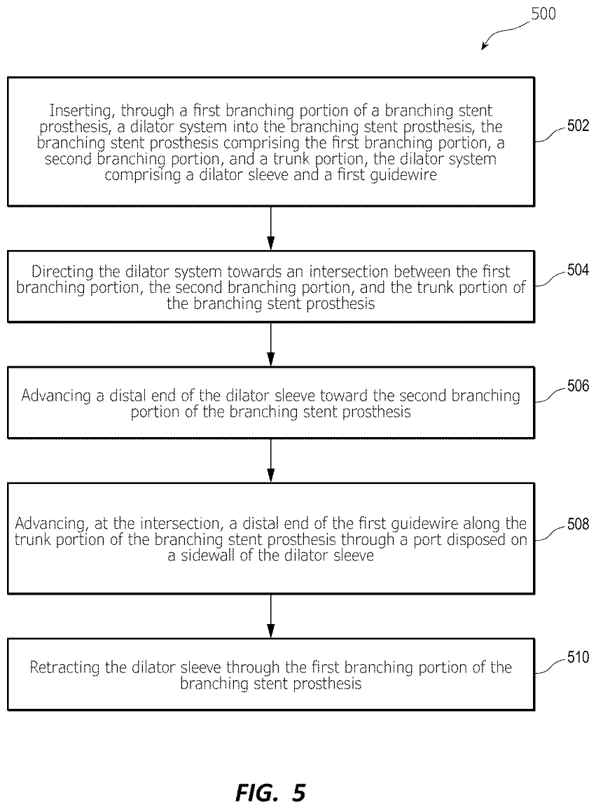

FIG. 5 illustrates a method 500, according to embodiments disclosed herein. The method 500 includes inserting, through a first branching portion of a branching stent prosthesis, a dilator system into the branching stent prosthesis, the branching stent prosthesis comprising the first branching portion, a second branching portion, and a trunk portion, the dilator system comprising a dilator and a first guidewire (block 502). The trunk portion, the first branching portion, and the second branching portion may be the trunk portion 108, the first branching portion 110, and the second branching portion 112 of the branching stent prosthesis 100 of FIGS. 1-4, or they may be other suitable portions of other branching stent prostheses. The dilator system may be the dual-wire lumen system 200 of FIG. 2, the dual-wire lumen system 400 of FIG. 4, or any other suitable system.

The method 500 further includes directing the dilator system towards an intersection between the first branching portion, the second branching portion, and the trunk portion of the branching stent prosthesis (block 504).

The method 500 further includes advancing a distal end of the dilator toward the second branching portion of the branching stent prosthesis (block 506).

The method 500 further includes advancing, at the intersection, a distal end of the first guidewire along the trunk portion of the branching stent prosthesis through a port disposed on a sidewall of the dilator (block 508). The port may be located at the distal end of the dilator. In this sense, the port may be located proximate to an actual distal endpoint of the dilator, such that there is a finite distance between the endpoint and the location of the port. In some embodiments, the first guidewire is configured to exit the dilator through the port and be advanced within the trunk portion of the branching stent prosthesis while the dilator is directed toward the second branching portion of the branching stent prosthesis.

The method 500 further includes retracting the dilator through the first branching portion of the branching stent prosthesis (block 510). The dilator may be configured to be retracted through the first branching portion of the branching stent prosthesis, along the second guidewire, and independently of the first guidewire. In other words, the first guidewire is left in place when the dilator is retracted. In some embodiments, the dilator is configured to produce minimal disturbance to the first guidewire as it is retracted. In some embodiments, the second guidewire may be a floss wire.

In some embodiments, the method 500 may further include running the dilator system along a second guidewire that extends between the first branching portion and the second branching portion of the branching stent prosthesis. The second guidewire may be the guidewire 102 of FIGS. 1-4, or it may be another wire that runs between the first and second branching portions of the branching stent prosthesis. For example, in one embodiment, the second guidewire may be a wire that is positioned within the branching stent prosthesis during or before deploying the branching stent prosthesis to a branching implant site. In other embodiments, the second guidewire may be a wire that is positioned within the branching stent prosthesis after it is deployed, for example as part of another procedure that a practitioner would perform. A portion of the second guidewire is disposed within the dilator.

In some embodiments, the dilator system further comprises a guide sheath which houses the dilator, the first guidewire, and a portion the second guidewire and wherein the method 500 further comprises advancing the guide sheath within the first branching portion along the second guidewire toward the intersection to advance the dilator and the first guidewire along the second guidewire.

In some embodiments, the method 500 is performed on a branching stent prosthesis that is already deployed at a branching implant site. Thus, the method 500 further comprises delivering the branching stent prosthesis to a branching implant site comprising a trunk, a first branch, and a second branch. In some embodiments, the branching implant site is located within a vascular system of a body, such as an aorto-illiac bifurcation in an arterial system.

Although the dilators described herein include a single port, in some embodiments a dilator may have more than one port. In some embodiments, the more than one ports may be located at different locations around the sidewall (e.g., they are aligned in a ring pattern), but each being at the same distance from the distal endpoint of the dilator. In other embodiments, the more than one ports may be located along a single line on the sidewall of the dilator, but each being at a different distance from the distal endpoint of the dilator. In other embodiments, the more than one ports may each be located at a different point both around and along the dilator. Such configurations may be utilized to reduce constraints relating to the process of advancing and directing the first guidewire from a first branching portion of a branching stent prosthesis to a trunk portion of the branching stent prosthesis.

Although examples depicted herein relate to installation or deployment of a guidewire from a first branching portion to a trunk portion of a branching stent prosthesis, in other embodiments, any methods described are applicable to deploying a guidewire between any two branches (out of the first branching portion, the second branching portion, and the trunk portion) by advancing the dilator system along a second guidewire extending between two other branches (out of the first branching portion, the second branching portion, and the trunk portion).

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in further embodiments, the feature can have a precisely perpendicular configuration.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A method comprising:
inserting, through a first branching portion of a branching stent prosthesis, a dilator system into the branching stent prosthesis, the branching stent prosthesis comprising the first branching portion, a second branching portion, and a trunk portion, the dilator system comprising a dilator and a first guidewire;
directing the dilator system towards an intersection between the first branching portion, the second branching portion, and the trunk portion of the branching stent prosthesis;
advancing a distal end of the dilator toward the second branching portion of the branching stent prosthesis;
advancing, at the intersection, a distal end of the first guidewire along the trunk portion of the branching stent prosthesis through a port disposed on a sidewall of the dilator; and
retracting the dilator through the first branching portion of the branching stent prosthesis.

2. The method of claim 1, wherein the port is disposed at the distal end of the dilator.

3. The method of claim 1, further comprising running the dilator system along a second guidewire that extends between the first branching portion and the second branching portion of the branching stent prosthesis.

4. The method of claim 1, wherein the dilator system further comprises a guide sheath which houses the dilator, the first guidewire, and a portion the second guidewire and wherein the method further comprises advancing the guide sheath within the first branching portion along the second guidewire toward the intersection to advance the dilator and the first guidewire along the second guidewire.

5. The method of claim 1, wherein a portion of the second guidewire is disposed within the dilator.

6. The method of claim 1, further comprising delivering the branching stent prosthesis to a branching implant site comprising a trunk, a first branch, and a second branch.

7. The method of claim 1, wherein the branching stent prosthesis is disposed at a branching implant site within a vascular system of a body.

8. A system comprising:
a branching stent prosthesis having a first branching portion, a second branching portion, and trunk portion;
a dilator comprising a port disposed on a sidewall of the dilator; and
a first guidewire configured to be advanced through the first branching portion and out the trunk portion of the branching stent prosthesis via the port of the dilator.

9. The system of claim 8, wherein the port is disposed at a distal end of the dilator.

10. The system of claim 8, further comprising:
a second guidewire extending between the first branching portion and the second branching portion of the branching stent prosthesis; and
a guide sheath housing the dilator, the first guidewire, and a portion of the second guidewire, the guide sheath to advance the dilator and the first guidewire along the second guidewire within the first branching portion of the branching stent prosthesis.

11. The system of claim 10, wherein the dilator and the first guidewire are advanced along the second guidewire towards an intersection between the first branching portion, the second branching portion, and the trunk portion of the branching stent prosthesis to direct the dilator toward the second branching portion of the branching stent prosthesis and to direct the first guidewire along the trunk portion of the branching stent prosthesis.

12. The system of claim 8, wherein the dilator is configured to be retracted through the first branching portion of the branching stent prosthesis.

13. The system of claim 8, further comprising a balloon disposed within the branching stent prosthesis to expand the branching stent prosthesis.

14. The system of claim 8, wherein the branching stent prosthesis is disposed at a branching implant site within a vascular system of a body.

15. A system for use with a branching stent prosthesis having a first branching portion, a second branching portion, and a trunk portion joined at an intersection, the system comprising:
a dilator comprising a port within a sidewall of the dilator;
a first guidewire disposed within the dilator; and
a second guidewire, wherein a portion of the second guidewire is disposed within the guide sheath.

16. The system of claim 15, further comprising a guide sheath configured to transport the first guidewire and the dilator along the second guidewire from the first branching portion of the branching stent prosthesis towards the intersection, wherein the first guidewire, the second guidewire, and the dilator are disposed within the guide sheath.

17. The system of claim 15, wherein the second guidewire extends between the first branching portion and the second branching portion of the branching stent prosthesis.

18. The system of claim 15, wherein at the intersection, the first guidewire is configured to exit the dilator through the port and be advanced within the trunk portion of the branching stent prosthesis and the dilator is directed toward to second branching portion of the branching stent prosthesis.

19. The system of claim 15, wherein the dilator is configured to be retracted through the first branching portion of the branching stent prosthesis independently of the first guidewire.

20. The system of claim 15, wherein the port is disposed at a distal end of the dilator.
